(19) United States
(12) Patent Application Publication
(10) Pub. No.: US 2019/0057766 A1
(43) Pub. Date: Feb. 21, 2019

(54) SYSTEM FOR MONITORING DIET

(71) Applicant: BOE Technology Group Co., Ltd., Beijing (CN)

(72) Inventors: Enhui Guan, Beijing (CN); Xinxin Mu, Beijing (CN); Shuo Chen, Beijing (CN); Lu Tong, Beijing (CN)

(21) Appl. No.: 16/021,539

(22) Filed: Jun. 28, 2018

(30) Foreign Application Priority Data
Aug. 21, 2017 (CN) 201710718117.7

(51) Int. Cl. G16H 20/60; A61B 5/00

(52) U.S. Cl. CPC G16H 20/60; G09B 19/0092; A61B 5/6897

(57) ABSTRACT

A system for monitoring diet. The system for monitoring diet includes: an information transmitter configured to transmit information of a food to be eaten in response to an operation of an eater taking out the food to be eaten; and an information processor configured to receive the information of the food from the information transmitter and to determine whether the food to be eaten meets requirements or not according to the information of the food to be eaten and a pre-stored information regarding the food that the eater needs to ingest.

### CROSS-REFERENCE TO RELATED APPLICATION

This application claims the priority benefit of Chinese Patent Application No. 201710718117.7 filed on Aug. 21, 2017 in the State Intellectual Property Office of China, the disclosure of which is incorporated herein by reference in entirety.

### TECHNICAL FIELD

Embodiments of the present disclosure relate to the field of smart system or smart living, and in particular, to a system for monitoring diet.

### BACKGROUND

The existing diet controlling methods are mainly designed for people who have a small amount of food intake or need to lose weight, and a quantitative recipe is made for them, so as to help them to control the diet.

### SUMMARY

An embodiment of the present disclosure provides a system for monitoring diet, comprising: an information transmitter configured to transmit information of a food to be eaten in response to an operation of an eater taking out the food to be eaten; and an information processor configured to receive the information of the food from the information transmitter and to determine whether the food to be eaten meets requirements or not according to the information of the food to be eaten and a pre-stored information regarding the food that the eater needs to ingest.

In a possible embodiment, the information processor comprises: an information reader configured to receive the information of the food to be eaten and to transmit the read information of the food; and a computing device configured to receive the read information of the food from the information reader and to determine whether the food to be eaten meets requirements or not according to the information of the food to be eaten and the pre-stored information regarding the food that the eater needs to ingest.

In a possible embodiment, in response to the information of the food comprising a weight of the food, the information processor is configured to determine whether the weight of the food to be eaten is within a weight range of the food that the eater needs to ingest or not according to the weight of the food to be eaten and a pre-stored range of the weight of the food that the eater needs to ingest.

In a possible embodiment, in response to the information of the food comprising the weight of the food, the computing device is configured to determine the weight of the food to be eaten according to the information of the food to be eaten, and to determine whether the weight of the food to be eaten is within a weight range of the food that the eater needs to ingest or not according to a pre-stored range of the weight of the food that the eater needs to ingest.

In a possible embodiment, in response to the information of the food comprising a content of a certain ingredient of the food, the information processor is configured to determine whether a weight of the certain ingredient of the food to be eaten is within a weight range of the certain ingredient of the food that the eater needs to ingest or not according to the weight of the certain ingredient of the food to be eaten and a pre-stored range of the weight of the certain ingredient of the food that the eater needs to ingest.

In a possible embodiment, in response to the information of the food comprising a content of a certain ingredient of the food, the computing device is configured to determine a weight of the certain ingredient of the food to be eaten according to the information of the food to be eaten, and determine whether the weight of the certain ingredient of the food to be eaten is within a weight range of the certain ingredient of the food that the eater needs to ingest or not according to a pre-stored range of the weight of the certain ingredient of the food that the eater needs to ingest.

In a possible embodiment, in response to the information of the food comprising a weight of the food and a content of a certain ingredient of the food, the computing device is configured to: determine the weight of the food to be eaten according to the information of the food to be eaten, and determine a weight of the certain ingredient of the food to be eaten according to the weight of the food to be eaten and the content of the certain ingredient of the food to be eaten; determine whether the weight of the food to be eaten is within a weight range of the food that the eater needs to ingest or not according to the weight of the food to be eaten and the weight of the food that the eater needs to ingest; and determine whether the weight of the certain ingredient of the food to be eaten is within a weight range of the certain ingredient of the food that the eater needs to ingest or not.

In a possible embodiment, the information processor further comprises a diet reminder configured to send a reminding signal when it is determined that the food to be eaten does not meet the requirements.

In a possible embodiment, the information processor further comprises a diet reminder configured to send a reminding signal when it is determined that the weight of the food to be eaten exceeds the weight range of the food that the eater needs to ingest or the weight of the certain ingredient of the food to be eaten exceeds the weight range of the certain ingredient of the food that the eater needs to ingest.

In a possible embodiment, the diet reminder is configured to: in response to the weight of the food to be eaten exceeding the weight range of the food that the eater needs to ingest, send a first reminding signal for reminding the eater that the weight of the food exceeds the weight range of the food that the eater needs to ingest; and in response to the weight of the certain ingredient of the food to be eaten exceeding the weight range of the certain ingredient of the food that the eater needs to ingest, send a second reminding signal for reminding the eater that the weight of the certain ingredient of the food exceeds the weight range of the certain ingredient of the food that the eater needs to ingest.

In a possible embodiment, in response to the information of the food comprising an expiration date of the food, the computing device is configured to: determine whether the food to be eaten is within the expiration date or not according to the expiration date of the food to be eaten, and send a warning signal for warning the eater that the food has expired in response to the food exceeding the expiration date.

In a possible embodiment, in response to the information of the food comprising species information of the food, the computing device is configured to: determine the weight of each species in the food to be eaten and a total weight of a certain ingredient of in the food to be eaten according to the information of the food to be eaten; determine a total weight of the food to be eaten by the eater according to the weights of each species in the food to be eaten, and determine whether the total weight of the food to be eaten is within a weight range of the food that the eater needs to ingest or not; and determine whether the total weight of the certain ingredient of the food to be eaten is within a weight range of the certain ingredient of the food that the eater needs to ingest or not.

In a possible embodiment, determining the weight of each species in the food to be eaten and the total weight of the certain ingredient of the food to be eaten according to the information of the food to be eaten comprises: determining the weight of each species in the food to be eaten according to the species information of the food to be eaten; and determining the total weight of the certain ingredient of the food to be eaten according to the weight of each species in the food and content of the certain ingredient of the species in the food.

In a possible embodiment, the system further comprises one or more storage containers for storing food.

In a possible embodiment, the information transmitter is disposed on the storage containers.

In a possible embodiment, each one of the storage containers comprises a sensor for sensing a change of the food in the one of the storage containers, and when it is sensed by the sensor that the food in the one of the storage containers changes, the sensor is configured to transmit a trigger signal to the information transmitter, and the information transmitter is configured to transmit the information of the food to be eaten to the information processor according to the trigger signal.

In a possible embodiment, the sensor is a weight sensor, and the weight sensor is configured to transmit the trigger signal to the information transmitter when it is sensed that a change of a weight of the food in the one of the storage containers is greater than a predetermined value.

In a possible embodiment, the system further comprises a camera and/or a scanner.

It is understood that other embodiments and configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 is a schematic structural view of a system for monitoring diet according to an embodiment of the present disclosure;

FIG. 2 is a schematic structural view showing a connection between an information transmitter and an information reader according to an embodiment of the present disclosure;

FIG. 3 is a schematic structural view of a storage container according to an embodiment of the present disclosure; and

FIG. 4 is a schematic structural view of a storage container according to another embodiment of the present disclosure.

### DETAILED DESCRIPTION OF EMBODIMENTS

It should be noted that specific details are set forth in the following description in order to fully understand the embodiments of the present disclosure. However, the embodiments of the present disclosure can be implemented in a variety of other manners other than those described herein, and modifications can be made to the present disclosure by those skilled in the art without departing from the spirit of the present disclosure. Therefore, the present disclosure is not limited to the specific embodiments disclosed below. For example, some terms are used throughout the specification and the claims to refer to particular components, it will be appreciated by those skilled in the art that hardware manufacturers may use different terms to name the same component. In the specification and the claims, the components are distinguished by their functions, rather than their names. As used throughout the specification and the claims, “comprise” is open-ended, and it should be interpreted as “including but not limited to”. The subsequent description of the specification refers to optional embodiments of the present disclosure, however, the description is intended to illustrate general principles of the present disclosure, but not intended to limit the scope of the present disclosure. The scope of the present disclosure is defined by the appended claims.

There are no solutions for monitoring diet for specific groups such as pregnant women or diabetic patients in the related art. Compared with ordinary people, it generally applies stricter dietary requirements for the specific groups such as pregnant women or diabetic patients, and they need more time periods for eating, and it is necessary to monitor intake of certain special elements in food, so as to ensure that eaters are in healthy diet. The embodiments of the present disclosure provide a system for monitoring diet to effectively monitor dietary condition of the eater to achieve healthy diet.

It should be noted that, in the embodiments of the present disclosure, a plurality of storage containers are preset, each storage container stores food, and then an information transmitter according to the embodiments of the present disclosure is disposed in the storage container so that the information transmitter can monitor the food in the storage container in real time. The capacity of each storage container can be designed according to dietary needs of the eaters, and it will not be specifically limited herein.

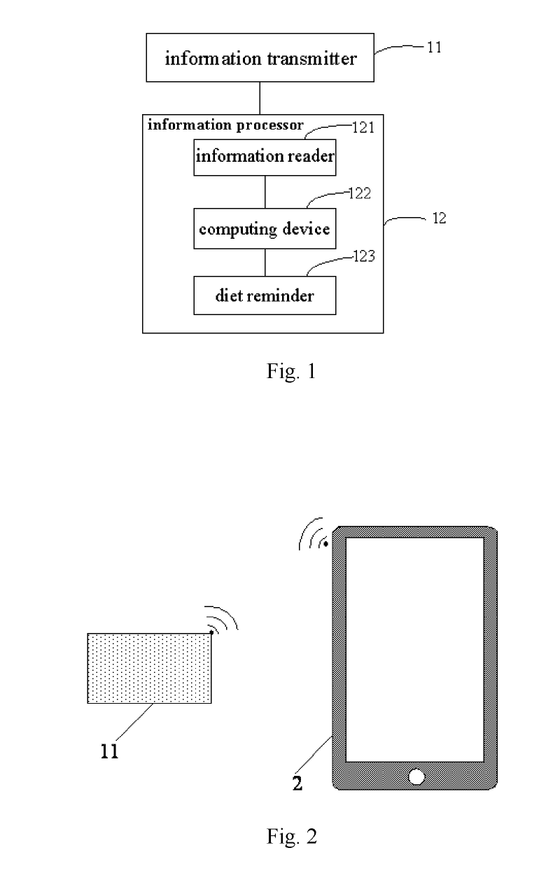

Referring to FIG. 1, an embodiment of the present disclosure provides a system for monitoring diet. The system includes an information transmitter 11 for transmitting information of a food to be eaten to an information processor 12 when an eater takes out the food to be eaten. The information of the food includes at least a weight of food and a content of a certain ingredient of the food.

The information transmitter according to the embodiment of the present disclosure may be disposed in a storage container for storing food. The storage container including the information transmitter is used for storing food, and when the eater takes out the food to be eaten from the storage container, the information transmitter transmits the information of the food to be eaten to the information processor. The information of the food to be eaten includes at least the weight of the food and the content of the certain ingredient of the food. The certain ingredient included in the information of the food may be set according to mineral elements of the food that the eater wants to control. If the eater is a diabetic patient, the certain ingredient may be set to glucose or the like. The certain ingredients are not specifically limited herein.

Specifically, a sensor, such as a weight sensor, may also be provided in the storage container. When the eater takes out the food to be eaten from the storage container, the weight of the food in the storage container changes, and the sensor senses the change, and then the information transmitter in the storage container transmits the changed value to the information processor. Herein, the changed value is the weight of the food to be eaten.

The system further includes an information processor 12 configured to determine whether the weight of the food to be eaten is within a weight range of the food that the eater needs to ingest or not and whether the weight of the certain ingredient in the food to be eaten is within a weight range of the certain ingredient of the food that the eater needs to ingest or not according to the information of the food to be eaten and a pre-stored range of the weight of the food that the eater needs to ingest (for example, the weight of the food that the eater needs to ingest within a predetermined period of time) and a pre-stored range of the weight of the certain ingredient of the food that the eater needs to ingest. If yes, it is determined that the food to be eaten meets requirements.

Specifically, the information processor in the embodiment of the present disclosure can implement multiply diet controls according to dietary requirements in every eating (or within a predetermined period of time) which are preset by the eater, for example, a weight range of the food that the eater needs to ingest each time, and a weight range of the certain ingredient of the food that the eater needs to ingest each time. Thus, the system for monitoring the diet monitors a dietary condition of the eater according to the dietary requirements.

In the system for monitoring the diet provided by the embodiment of the present disclosure, the information transmitter is configured to transmit the information of the food to be eaten to the information processor when the eater takes out the food to be eaten. The information of the food includes at least the weight of food and the content of the certain ingredient of the food; the information processor is configured to determine whether the weight of the food to be eaten is within the weight range of the food that the eater needs to ingest and the weight of the certain ingredient in the food to be eaten is within the weight range of the certain ingredient of the food that the eater needs to ingest or not according to the information of the food to be eaten and the pre-stored range of the weight of the food that the eater needs to ingest and the pre-stored range of the weight of the certain ingredient of the food that the eater needs to ingest; if yes, it is determined that the food to be eaten meets requirements or the dietary condition of the eater is healthy. Therefore, by means of the system for monitoring the diet provided by the embodiment of the present disclosure, it is monitored whether the weight of the food to be eaten is within the weight range of the food that the eater needs to ingest and the weight of the certain ingredient in the food to be eaten is within the weight range of the certain ingredient of the food that the eater needs to ingest or not before the eater eats the food; if yes, it is determined that the food to be eaten meets requirements or the dietary condition of the eater is healthy, so as to achieve the purpose of monitoring the diet. Herein, the certain ingredient can be set according to specific needs of the eater, such as needs on intake of glucose for a diabetic patient, needs on intake of certain mineral element for a pregnant woman, or the like. It can be seen that the system according to the embodiment of the present disclosure can effectively monitor the dietary condition of the eater and achieve the healthy diet.

In a specific embodiment, in the system according to the above-described embodiment of the present disclosure, referring to FIG. 1, the information processor 12 includes: an information reader 121 configured to receive the information of the food to be eaten and transmit the read information of the food; and a computing device 122 configured to receive the read information of the food from the information reader 121 and to determine the weight of the food to be eaten and the weight of the certain ingredient of the food to be eaten according to the information of the food to be eaten; and to determine whether the weight of the food to be eaten is within the weight range of the food that the eater needs to ingest or not and whether the weight of the certain ingredient of the food to be eaten is within the weight range of the certain ingredient of the food that the eater needs to ingest or not according to the pre-stored weight range of the food that the eater needs to ingest and the pre-stored weight range of the certain ingredient of the food that the eater needs to ingest.

If they are within the pre-stored weight ranges, it is determined that the food to be eaten meets requirements or the dietary condition of the eater is healthy.

Specifically, the information transmitter is connected to the information reader in the embodiment of the present disclosure. In an example, the information transmitter and the information reader can be connected to each other by wireless, Bluetooth or the like. The information transmitter updates the information of the food in the storage container in real time. If the food in the storage container changes, the information transmitter transmits the changed weight of the food and the content of the certain ingredient to the information reader.

In a specific embodiment, in the system according to the above-described embodiment of the present disclosure, the computing device 122 is specifically configured to: determine the weight of the food to be eaten according to the information of the food to be eaten, and determine a weight of the certain ingredient of the food to be eaten according to the weight of the food to be eaten and the content of the certain ingredient of the food to be eaten; determine whether the weight of the food to be eaten is within a weight range of the food that the eater needs to ingest or not according to the weight of the food to be eaten and the weight of the food that the eater needs to ingest; if yes, determine whether the weight of the certain ingredient of the food to be eaten is within a weight range of the certain ingredient of the food that the eater needs to ingest or not; if yes, it is determined that the food to be eaten meets requirements or the dietary condition of the eater is healthy.

Specifically, it is detected whether or not the weight of the food and the weight of the certain ingredient of the food are within the dietary requirements of the eater, the information transmitter stores the information of the food in the storage container, including the weight of the food and the content of the certain ingredient of the food. If the eater takes out a portion of the food from the storage container, the information transmitter transmits the information of the food in the storage container to the information reader, and the information is the information regarding change of the weight of the food in the storage container, that is, the weight of the food that is taken out. For example, it is determined that the weight of the food to be eaten by the eater is X3, the weight of the food that needs to be ingested ranges from X4(1) to X4(2), the content of the certain ingredient of the food is X6, and the weight of the certain ingredient of the food that needs to be ingested ranges from X5(1) to X5(2). Then it is determined whether X3 is within the range of X4(1) to X4(2); if yes, it is determined that the weight of the food to be eaten by the eater is within the weight range of the food that the eater needs to ingest. A product of X3 and X6 is determined at first, then it is determined whether X3*X6 is within the range of X5(1) to X5(2); if yes, it is determined that the weight of the certain ingredient of the food to be eaten is within the weight range of the certain ingredient of the food that the eater needs to ingest.

In a specific embodiment, in the system according to the above-described embodiment of the present disclosure, referring to FIG. 1, the information processor 12 further includes a diet reminder 123 configured to send a reminding signal when it is determined that the weight of the food to be eaten exceeds the weight range of the food that the eater needs to ingest or the weight of the certain ingredient of the food to be eaten exceeds the weight range of the certain ingredient of the food that the eater needs to ingest. Specifically, in order to further remind the eater that the food does not meet the dietary requirements, a reminding signal may be issued.

In a specific embodiment, in the system according to the above-described embodiment of the present disclosure, the diet reminder 123 is configured to: in response to the weight of the food to be eaten by the eater exceeding the weight range of the food that the eater needs to ingest, send a first reminding signal for reminding the eater that the weight of the food exceeds the weight range of the food that the eater needs to ingest; and in response to the product of the weight of the food to be eaten and the content of the certain ingredient of the food to be eaten exceeding the weight range of the certain ingredient of the food that the eater needs to ingest, send a second reminding signal for reminding the eater that the weight of the certain ingredient of the food exceeds the weight range of the certain ingredient of the food that the eater needs to ingest. Herein, the reminding signal can be sent by means of sound, light, or the like, which is not specifically limited herein.

For example, if X3 is not within the range of X4(1) to X4(2), then it is determined that the weight of the food to be eaten by the eater is not within the weight range of the food that the eater needs to ingest, then a first reminding signal is sent to remind the eater that the weight of the food exceeds the weight range of the food that the eater needs to ingest. If X3 is less than X4(1), then it is determined that the weight of the food is less than the weight range of the food that the eater needs to ingest, then a detailed reminding signal is further sent to remind the eater that the weight of the food is less than the weight of the food that the eater needs to ingest and the food can be eaten. If X3 is greater than X4(2), then it is determined that the weight of the food is greater than the weight of the food that the eater needs to ingest, then a detailed reminding signal is further sent to remind the eater that the weight of the food is greater than the weight of the food that the eater needs to ingest and the food cannot be eaten. Further, when it is determined that the product of X3 and X6 is not within the range of X5(1) to X5(2), a second reminding signal is sent to remind the eater that the food to be eaten by the eater does not satisfy the weight range of the certain ingredient of the food that the eater needs to ingest. If X3*X6 is less than X5(1), then a detailed reminding signal is further sent to remind the eater that the weight of the certain ingredient of the food to be eaten is less than the weight range of the certain ingredient of the food that the eater needs to ingest and the food can be eaten. If X3*X6 is greater than X5(2), then a detailed reminding signal is further sent to remind the eater that the weight of the certain ingredient of the food to be eaten is greater than the weight range of the certain ingredient of the food that the eater needs to ingest and the food cannot be eaten.

In a specific embodiment, in the system according to the above-described embodiment of the present disclosure, in order to ensure freshness of the food stored in the storage container, it is necessary to further detect whether the food to be eaten does not exceed an expiration date before the food is supplied to the eater. The information of the food also includes the expiration date of the food, and the computing device is also configured to, before determining the weight of the food to be eaten and the weight of the certain ingredient of the food to be eaten, determine whether the food to be eaten is within the expiration date or not according to the expiration date of the food to be eaten; if no, then a warning signal for warning the eater that the food has expired is sent.

For example, after the information reader sends the information of the food to the computing device, the computing device at first detects the freshness of the food. Firstly, today's date is determined as X1, secondly, the best edible date of the food is determined as X2, and thirdly, X1 is compared with X2. If it is determined that X15 X2, then the food is fresh and edible, or otherwise the food is inedible. Alternatively, after it is determined that the food is inedible, a warning signal is sent to the eater. The warning signal may be sent by means of various manners, for example, a sound may be played to warn the eater, or warning information may be displayed on a display device to warn the eater, and they will not be specifically limited herein.

It should be noted that, the storage container in the embodiments of the present disclosure can store food in terms of different specieses, for example, the same species of food is placed in one and the same storage container, and different specieses of food are respectively placed in different storage containers respectively; or alternatively, different specieses of food are placed in one and the same storage container for storage. In the letter case, the information of the food stored in the information transmitter also includes the species of the food.

In a specific embodiment, in the system according to the above-described embodiment of the present disclosure, the information of the food to be eaten also includes the species of the food; the computing device is configured to: determine the weight of each species in the food to be eaten and a total weight of the certain ingredient of the food to be eaten according to the information of the food to be eaten; determine a total weight of the food to be eaten by the eater according to the weight of each species in the food to be eaten, and determine whether the total weight of the food to be eaten is within a weight range of the food that the eater needs to ingest or not; if yes, determine whether the total weight of the certain ingredient of the food to be eaten is within a weight range of the certain ingredient of the food that the eater needs to ingest or not; if yes, it is determined that the food to be eaten meets requirements or the dietary condition of the eater is healthy.

Specifically, the information of the food stored by the information transmitter in the embodiments of the present disclosure includes the species, the weight, the content of the certain ingredient, and the expiration date of the food.

If the food that the eater takes out from the storage container includes not only one species of food, then the information transmitter also stores the species of the food and the weight of such species of food. The information transmitter in the embodiment of the present disclosure also transmits the species of the food and the weight of such species of the food when transmitting the information of the food. Specifically, if there are many foods stored in the storage container and the eater only takes out some foods, only one species of foods may be taken out each time, so as to further determine the weight of each species of food taken out by the eater. For example, a first species of food is taken out at the first time, and a second species of foods is taken out at the second time, so that it is convenient for the information transmitter to store the weight of each species of food and transmit it to the information reader. For example, a camera may be installed beside the storage container. As an example, it is determined that the first species of food is taken out by the eater at the first time by means of image recognition technology using the camera, and then the weight of the food in the storage container is determined by means of the sensor in the storage container, thereby further determining the weight of the first species of food; then it is determined that the second species of food is taken out by the eater at the second time by means of image recognition technology using the camera, and then the weight of the food in the storage container is determined by means of the sensor in the storage container, thereby further determining the weight of the second species of food; and so on.

For example, if two specieses of food are stored in a storage container, the weight of the first species of food taken out by the eater is Y1X1, the weight of the second species of food is Y2X1, the content of the certain ingredient in the first species of food is Y1X2, and the content of the certain ingredient in the second species of food is Y2X2, then the weight of the food to be eaten by the eater is Y1X1+Y2X1, and the weight of the certain ingredient in the food to be eaten by the eater is Y1X1*Y1X2+Y2X1*Y2X2. If the weight range of the food that the eater needs to ingest is from X4(1) to X4(2), and the weight range of the certain ingredient of the food that the eater needs to ingest is from X5(1) to X5(2), then it is further determined whether Y1X1+Y2X1 is in the range of X4(1) to X4(2) and whether Y1X2+Y2X1*Y2X2 is in the range of X5(1) to X5(2) or not.

It should be noted that, in the embodiments of the present disclosure, two specieses of food are taken as an example to illustrate how to determine whether the food to be eaten meets the dietary requirements, but more species of food are possible. When there are more than two specieses of foods, they may be determined in the same manner as described above, and they will not be repeatedly described herein.

The information reader, the computing device and the diet reminder in the embodiments of the present disclosure may be installed in a mobile phone or a computer, as shown in FIG. 2. If the information reader, the computing device and the diet reminder are installed in a mobile phone, the information transmitter 11 may be connected with the mobile phone 2 by wireless, so that the information transmitter transmits the information of the food in the storage container to the computing device through the information reader in real time.

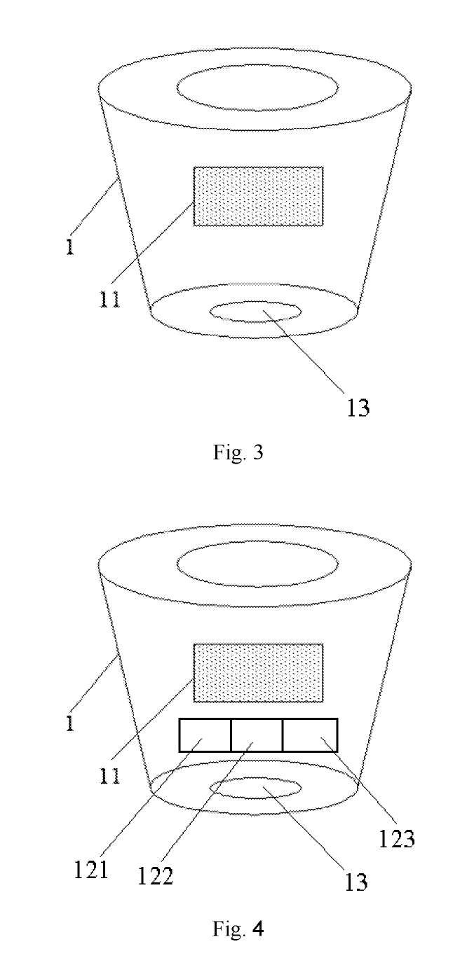

Based on the same inventive concept, an embodiment of the present disclosure further provides a storage container. As shown in FIG. 3, the storage container 1 includes an information transmitter 11 configured to transmit information of a food to be eaten to an information processor when an eater takes out the food to be eaten. The information of the food includes at least a weight of food and a content of a certain ingredient of the food. Therefore, the storage container according to the embodiment of the present disclosure monitors the weight, the expiration date, the content of the certain ingredient, and the species of the food in the storage container in real time through the information transmitter. A sensor may be further provided in the storage container to monitor the weight of the food in real time. The storage container may be in a shape of cylinder, cuboid, or the like, which is not specifically limited herein.

Further, as shown in FIG. 3, the storage container 1 includes a sensor 13. The sensor 13 may be disposed at the bottom of the container for sensing the change of the food in the storage container 1. For example, the sensor 13 may be a weight sensor for sensing the weight of the food in the storage container 1. When it is sensed by the weight sensor that the weight of the food in the storage container 1 changes, a trigger signal is sent to the information transmitter 11, then the information transmitter 11 transmits the information of the food to be eaten to the information processor. That is to say, a sensed result of the weight sensor (i.e., the change of the weight of the food in the storage container 1) may be used as a basis for determining whether the eater takes out the food to be eaten. Alternatively, if it is sensed by the weight sensor that the weight change of the food in the storage container 1 is greater than a predetermined value, a trigger signal is sent to the information transmitter 11.

Alternatively, the sensor 13 may also be another type of sensor as long as it can sense the change of the food in the storage container 1. As an example, a counter, a count button and a display screen are provided on the storage container 1. The display screen is configured to display information such as name and/or quantity of the food stored in the storage container 1. The counter stores the quantity of the food stored in the storage container 1, and the count button can increase or decrease count value of the counter gradually. In this way, the storage container 1 can work in the following manner: after the food is placed in the storage container 1, the count value of the counter is adjusted by manually pressing the count button until it is equal to the quantity of the food in the storage container 1; when the eater takes out the food in the storage container 1, he/she manually presses the count button to decrement the count value of the counter. Further, when the count value of the counter decreases, the information transmitter 11 transmits the information of the food to be eaten to the information processor, that is, a change in the count value of the counter is used as a basis for determining whether the eater takes out the food to be eaten.

Further, a scanner is provided on the storage container 1 or in the vicinity of the storage container 1. The scanner can scan for example a two-dimensional code or a bar code. Accordingly, a two-dimensional code or a bar code may be provided on a food package. In this way, when the eater takes out the food in the storage container 1, the two-dimensional code or the bar code on the food package may be scanned by the scanner, and thereby the scanner obtains the information of the food, and then the scanner sends a trigger signal to the information transmitter 11 and the information of the food is sent to the information transmitter, and then the information transmitter 11 transmits the information of the food to be eaten to the information processor.

Alternatively, a camera may be provided on the storage container 1 or in the vicinity of the storage container 1. The camera collects an image of the food in the storage container 1 taken by the eater, and transmits the image to the information processor 12, and then the computing device 122 of the information processor 12 obtains the information of the food through image recognition technology.

FIG. 4 is a schematic structural view of a storage container according to another embodiment of the present disclosure. This embodiment is distinguished from the embodiment shown in FIG. 3 in that the information reader 121, the computing device 122 and the diet reminder 123 of the information processor are provided on the storage container 1. In this way, the eater's diet can be monitored without a mobile phone or a computer.

In summary, the embodiments of the present disclosure provide a system for monitoring diet. The information transmitter is configured to transmit the information of the food to be eaten to the information processor when the eater takes out the food to be eaten. The information of the food includes the weight of food and the content of the certain ingredient of the food; the information processor is configured to determine whether the weight of the food to be eaten is within the weight range of the food that the eater needs to ingest and the weight of the certain ingredient in the food to be eaten is within the weight range of the certain ingredient of the food that the eater needs to ingest or not according to the information of the food to be eaten and the pre-stored range of the weight of the food that the eater needs to ingest and the pre-stored range of the weight of the certain ingredient of the food that the eater needs to ingest; if yes, it is determined that the food to be eaten meets requirements or the dietary condition of the eater is healthy. Therefore, for the system for monitoring the diet provided by the embodiment of the present disclosure, it is monitored whether the weight of the food to be eaten is within the weight range of the food that the eater needs to ingest and the weight of the certain ingredient in the food to be eaten is within the weight range of the certain ingredient of the food that the eater needs to ingest or not, before the eater eats the food; if yes, it is determined that the food to be eaten meets requirements or the dietary condition of the eater is healthy, so as to achieve the purpose of monitoring the diet. Herein, the certain ingredient can be set according to specific needs of the eater, such as needs on intake of glucose for a diabetic patient, needs on intake of certain mineral element for a pregnant woman, or the like. It can be seen that the system according to the embodiment of the present disclosure can effectively monitor the dietary condition of the eater and achieve the healthy diet.

It will be apparent for those skilled in the art that various modifications and variations can be made to the embodiments of the present disclosure without departing from the spirit and scope of the present disclosure. Thus, if these modifications and variations of the present disclosure fall within the scope of the claims of the present disclosure and equivalent thereof, the present disclosure is also intended to include these modifications and variations.

What is claimed is:

1. A system for monitoring diet, comprising:
an information transmitter configured to transmit information of a food to be eaten in response to an operation of an eater taking out the food to be eaten; and
an information processor configured to receive the information of the food from the information transmitter and to determine whether the food to be eaten meets requirements or not according to the information of the food to be eaten and a pre-stored information regarding the food that the eater needs to ingest.

2. The system according to claim 1, wherein the information processor comprises:
an information reader configured to receive the information of the food to be eaten and to transmit the read information of the food; and
a computing device configured to receive the read information of the food from the information reader and to determine whether the food to be eaten meets requirements or not according to the information of the food to be eaten and the pre-stored information regarding the food that the eater needs to ingest.

3. The system according to claim 1, wherein, in response to the information of the food comprising a weight of the food, the information processor is configured to determine whether the weight of the food to be eaten is within a weight range of the food that the eater needs to ingest or not according to the weight of the food to be eaten and a pre-stored range of the weight of the food that the eater needs to ingest.

4. The system according to claim 2, wherein, in response to the information of the food comprising the weight of the food, the computing device is configured to determine the weight of the food to be eaten according to the information of the food to be eaten, and to determine whether the weight of the food to be eaten is within a weight range of the food that the eater needs to ingest or not according to a pre-stored range of the weight of the food that the eater needs to ingest.

5. The system according to claim 1, wherein, in response to the information of the food comprising a content of a certain ingredient of the food, the information processor is configured to determine whether a weight of the certain ingredient of the food to be eaten is within a weight range of the certain ingredient of the food that the eater needs to ingest or not according to the weight of the certain ingredient of the food to be eaten and a pre-stored range of the weight of the certain ingredient of the food that the eater needs to ingest.

6. The system according to claim 2, wherein, in response to the information of the food comprising a content of a certain ingredient of the food, the computing device is configured to determine a weight of the certain ingredient of the food to be eaten according to the information of the food to be eaten, and determine whether the weight of the certain ingredient of the food to be eaten is within a weight range of the certain ingredient of the food that the eater needs to ingest or not according to a pre-stored range of the weight of the certain ingredient of the food that the eater needs to ingest.

7. The system according to claim 2, wherein, in response to the information of the food comprising a weight of the food and a content of a certain ingredient of the food, the computing device is configured to:
determine the weight of the food to be eaten according to the information of the food to be eaten, and determine a weight of the certain ingredient of the food to be eaten according to the weight of the food to be eaten and the content of the certain ingredient of the food to be eaten;
determine whether the weight of the food to be eaten is within a weight range of the food that the eater needs to ingest or not according to the weight of the food to be eaten and the weight of the food that the eater needs to ingest; and
determine whether the weight of the certain ingredient of the food to be eaten is within a weight range of the certain ingredient of the food that the eater needs to ingest or not.

8. The system according to claim 2, wherein the information processor further comprises a diet reminder configured to send a reminding signal when it is determined that the food to be eaten does not meet the requirements.

9. The system according to claim 7, wherein the information processor further comprises a diet reminder configured to send a reminding signal when it is determined that the weight of the food to be eaten exceeds the weight range of the food that the eater needs to ingest or the weight of the certain ingredient of the food to be eaten exceeds the weight range of the certain ingredient of the food that the eater needs to ingest.

10. The system according to claim 9, wherein the diet reminder is configured to:
in response to the weight of the food to be eaten exceeding the weight range of the food that the eater needs to ingest, send a first reminding signal for reminding the eater that the weight of the food exceeds the weight range of the food that the eater needs to ingest; and
in response to the weight of the certain ingredient of the food to be eaten exceeding the weight range of the certain ingredient of the food that the eater needs to ingest, send a second reminding signal for reminding the eater that the weight of the certain ingredient of the food exceeds the weight range of the certain ingredient of the food that the eater needs to ingest.

11. The system according to claim 2, wherein, in response to the information of the food comprising an expiration date of the food, the computing device is configured to:
determine whether the food to be eaten is within the expiration date or not according to the expiration date of the food to be eaten, and
send a warning signal for warning the eater that the food has expired in response to the food exceeding the expiration date.

12. The system according to claim 2, wherein, in response to the information of the food comprising species information of the food, the computing device is configured to:
determine the weight of each species in the food to be eaten and a total weight of a certain ingredient of in the food to be eaten according to the information of the food to be eaten;
determine a total weight of the food to be eaten by the eater according to the weights of each species in the food to be eaten, and determine whether the total weight of the food to be eaten is within a weight range of the food that the eater needs to ingest or not; and
determine whether the total weight of the certain ingredient of the food to be eaten is within a weight range of the certain ingredient of the food that the eater needs to ingest or not.

13. The system according to claim 12, wherein determining the weight of each species in the food to be eaten and the total weight of the certain ingredient of the food to be eaten according to the information of the food to be eaten comprises:
determining the weight of each species in the food to be eaten according to the species information of the food to be eaten; and
determining the total weight of the certain ingredient of the food to be eaten according to the weight of each species in the food and content of the certain ingredient of the species in the food.

14. The system according to claim 1, further comprising one or more storage containers for storing food.

15. The system according to claim 14, wherein the information transmitter is disposed on the storage containers.

16. The system according to claim 14, wherein each one of the storage containers comprises a sensor for sensing a change of the food in the one of the storage containers, and when it is sensed by the sensor that the food in the one of the storage containers changes, the sensor is configured to transmit a trigger signal to the information transmitter, and the information transmitter is configured to transmit the information of the food to be eaten to the information processor according to the trigger signal.

17. The system according to claim 16, wherein the sensor is a weight sensor, and the weight sensor is configured to transmit the trigger signal to the information transmitter when it is sensed that a change of a weight of the food in the one of the storage containers is greater than a predetermined value.

18. The system according to claim 1, further comprising a camera and/or a scanner.
